(19) United States
(12) Patent Application Publication
(10) Pub. No.: US 2016/0374599 A1
(43) Pub. Date: Dec. 29, 2016

(54) Method and apparatus for non-invesive blood glucose monitoring system

(71) Applicant: Joseph Ralph Frattarola, Ada, OH (US)

(72) Inventor: Joseph Ralph Frattarola, Ada, OH (US)

(21) Appl. No.: 14/747,636

(22) Filed: Jun. 23, 2015

(51) Int. Cl. A61B 5/145; A61B 8/08; A61B 5/107; A61B 8/00

(52) U.S. Cl. CPC A61B 5/14532; A61B 8/42; A61B 8/5207; A61B 8/58; A61B 5/1075; A61B 8/54

(57) ABSTRACT

A new method and apparatus is provided for as a wearable, self-contained, non-invasive device for the instantaneous measurement of blood glucose in an individual. This invention uses the measurement and comparison of ultrasound wave velocities to determine the instant value of glucose concentration in the individual. In particular, it uses ultrasound waveforms in the “A” operational mode, depth determination, rather than typical imaging operational modes. Two distinct and different ultrasound frequencies are transmitted into the body and their total propagation times, from insert to bone-reflected echo return signal, are timed to calculate the signal dependent velocities affected by glucose. It is applicable to all forms of human body conditions, regardless of size, weight, age or health condition. It can be adapted for use in a closed loop system for insulin delivery on an “as-needed basis.”

### BACKGROUND OF THE INVENTION

There are 16 million diabetics in the USA alone and another 16 million in the rest of the world. Diabetes is a chronic disease brought on by the inability of the body's pancreas to produce insulin. Insulin counters the amount of glucose, sugar, entering the bloodstream as naturally produced by the body. It is the control of insulin production by the pancreas that balances the glucose level in the blood. Diabetics must control their blood glucose level by injections of insulin relative to daily multiple testings of their blood glucose levels. There is no cure for diabetes, only lifetime control.

Today's personal blood glucose monitoring systems consist of a skin-puncturing device, chemically treated test strips and a test strip measurement reader. Diabetics puncture their skin, usually fingertip, with a spring-loaded small, thin needle, lance, and then force blood out through the puncture hole. This blood sample is then placed on the test strip and inserted into the measurement reader. Interaction of the blood glucose and test strip chemical results in altering the color property of the strip. The reader performs a measurement whereby optical density, dark color, indicates a larger concentration of glucose; therefore, diabetics must maintain a stockpile of strips for their continuing multiple testings per day. A diabetic must preform such tests several times each day for assessment of insulin need. The non-reusable, disposable components are the lance and the test strips. Each quarter the reader must be calibrated by using a test solution with a test strip to simulate a known blood glucose value equivalent. This control solution is also disposable.

There are other methods proposed to measure real-time blood glucose levels in the body. Among these, only infrared light wave technology promises a non-invasive approach. Another is a wearable patch apparatus that permits multiple blood glucose readings on an automatic time-period basis. This device can only be worn for a short time as it demands a constant invasive penetration of a needle into the skin, presenting a risk of infection.

This system utilizes ultrasound technology to determine major parameters for the calculation of blood glucose values. Unlike electromagnetic radio frequency waves, ultrasound waves are pressure waves. Ultrasound wave velocities are directly proportional to the Bulk Modulus of the conducting medium, in this case body tissue containing blood.

### SUMMARY OF THE INVENTION

Ultrasound waves travel through a medium at a velocity directly proportional to the Bulk Modulus of that conducting medium. This invention is based upon the measurements of two different ultrasound frequency waveform propagation times for the determination of their respective velocities for subsequent calculation of blood glucose concentrations in the body.

In the preferred embodiment of the invention, one dual frequency ultrasound transducer is described; alternatively, two separate ultrasound transducers of differing frequencies can be used in a coplanar adjacent arrangement. Ultrasound transducers provide both a signal emitter and a signal sensor/collector. A transducer placed against the skin introduces a pressure wave signal into the body whereupon the wrist bone returns an echo signal for transducer detection. This waveform propagation time is precisely measured by a sub-nanosecond crystal-controlled electronic clock timing and used in combination with the distance traveled to calculate its velocity.

The invention utilizes two different ultrasound frequencies, one in the 1-5 MHz range and another in the 30-50 MHz range (e.g., 2 MHz and 30 MHz). Frequencies in the lower range produce waveform velocities that are directly proportionately dependent upon the medium's Bulk Modulus. Higher range frequency velocities are relatively independent and unaffected by the medium's Bulk Modulus due to the “Skin Effect.”

The “Skin Effect” is the tendency of high frequency energy to concentrate near the outer edge, or surface, of a conductor, instead of moving uniformly over the entire cross-sectional area of the conductor. The higher the frequency the greater the tendency for this effect to occur. The propagation of ultrasound waves, longitudinal pressure waves, through a conductor is governed by the Bulk Modulus of that conductor. The glucose properties of stiffness and elasticity determine ultrasound waveform velocity through the conductor. In the case of ultrahigh frequency wavelengths, 30-50 MHz, the Bulk Modulus has insufficient time to respond to the rapid changes in the ultrasound pressure wave; thus, the wave energy is skirted around those glucose molecules yielding an independent base velocity. The degree of effectiveness on ultrasound wave velocity due to “Skin Effect” varies from person to person. A supplementary Probe permits measurement of this base velocity.

It is the precise measurement of the two different ultrasound velocities that allows for the calculation of blood glucose concentration values. The dependent velocity value is used as a parameter for comparison of laboratory-prepared samples of blood glucose combinations over a range of 30-500 mg/dl as shown in Diagram 5.

### DETAILED DESCRIPTION OF THE INVENTION

Basis for the invention resides in the physical properties and dynamic interactions of ultrasound waves within human tissue. Parameters of distance, signal propagation times and wave velocity of specific frequencies are measured for determination of blood glucose values.

Two ultrasound frequencies that are significant to the invention are a high frequency 30-50 MHz termed A and a lower ultrasound frequency 2-5 MHz termed B. Ultrasound velocities of frequency A are independent of blood glucose levels due to the “Skin Effect” property of high frequency waveforms. Ultrasound velocities of frequency B are directly dependent upon blood glucose levels.

This invention consists of two major devices, a Probe (Diagram 2) and a main System Unit (Diagram 1). The Probe is a subsystem external to the main System Unit and is connected to it by a USB cable. All control electronic signals, timing, calculations, data storage, display and power source are configured within the main System Unit. Total system package is envisioned as a wearable wrist band apparatus as shown in Diagram 1. The back face of the main System Unit contains an output port for the ultrasound transducer, positioned above the package for firm contact with the skin. Both configurations, dual frequency transducer and two separate transducers, are shown in Diagram 1, FIGS. 1C and 1D. Also shown is the body temperature sensor pad. Temperature values affect the frequency waveform velocities as shown in Diagram 5.

The front face of the main System Unit contains a Microelectronics Platform (Diagrams 3 and 4) supporting a four-digit digital display, side-mounted system user function controls, a USB port, manual TEST button (activates the user manual test) and all timing and calculating functions. A side-mounted USB connection permits communication with the Probe unit and other external devices (e.g., computer). Three rotary push-button switches allow for the control of Time, Auto Test and Mode functions. Time readings are displayed on the four-digit digital LCD display and set with the Time switch. Auto Test allows the system to automatically perform blood glucose tests on a continuing preset periodic schedule. Mode functions are: Probe, data recall, testing and data out. Power source, button battery, is contained within the main System Unit.

Since the high frequency “Skin Effect” differs from person to person, its individual base velocity (V₀) must first be determined. This is accomplished with the external Probe. Diagram 2 illustrates the components of the Probe subsystem.

The Probe is a sliding apparatus that compresses its jaws onto skin tissue (e.g., an earlobe). One jaw face holds a transducer, and the other is used as the reflector plate for the ultrasound return signal. Within the Probe package is an electronic digital caliper module for measurement of the human tissue/thickness between jaws, providing the value of D. Its transducer receives a pulse frequency A of 30-50 MHz from the main System Unit through the USB cable. This signal frequency passes through the skin tissue and is reflected back to the transducer and timed by the main System Unit sub-nanosecond clock. This action provides the values of D (distance) and T (time) for the calculation of its base velocity (V₀).

V₀=D/2T

This base velocity is derived from the independent frequency A waveform and establishes an individual's parameter for long-term testing utility. This value of base velocity (V₀) is stored in system memory for subsequent utility.

System Operation

Initial setup of the system requires the establishment of the base velocity (V₀) as previously described. This procedure should be performed one or two times per year, or whenever necessitated by metabolic changes. A test parameter standard is further established for ongoing comparison of Probe-derived V_{0.}

Testing starts automatically or by manual action, depending upon user settings. The transducer(s) begin(s) the process by emitting a 30-50 MHz signal (A) into the skin at the wrist location. The velocity of this frequency signal, previously established, is V₀, base velocity. Signal propagation time (tₐ) is determined by the sub-nanosecond system clock. The value of dₐ is then calculated.

dₐ=V₀tₐ 2

This value is stored in memory. Immediately following this process, the transducer (or second unit) emits its 2-5 MHz ultrasound frequency pulse signal (B). Its total propagation time (t_{b}) is measured and used with dₐ to determine its blood glucose dependent velocity.

v_{b}=dₐ/t_{b}

It is this glucose dependent ultrasound waveform velocity that is used to match the laboratory matrix table samples (Diagram 5) to provide an instantaneous blood glucose value in the body. Diagrams 3 and 4 depict a typical state-of-the-art microelectronics configuration for the control, timing, storage, external communication and calculations system operation. Diagrams 3 and 4 illustrate the system operation for the single dual frequency transducer configuration and the two separate transducer configurations, respectively.

### BRIEF DESCRIPTION OF DRAWINGS

Diagram 1, FIG. 1A, shows a wearable package of the main System Unit.

FIG. 1B, shows the front face of the main System Unit with its control functions, output USB port, display and TEST button.

FIG. 1C, shows the rear face of the main System Unit, housing the dual frequency ultrasound transducer and temperature sensor.

FIG. 1D, shows the rear face of the main System Unit, housing the two separate single frequency transducers (A and B) and temperature sensor.

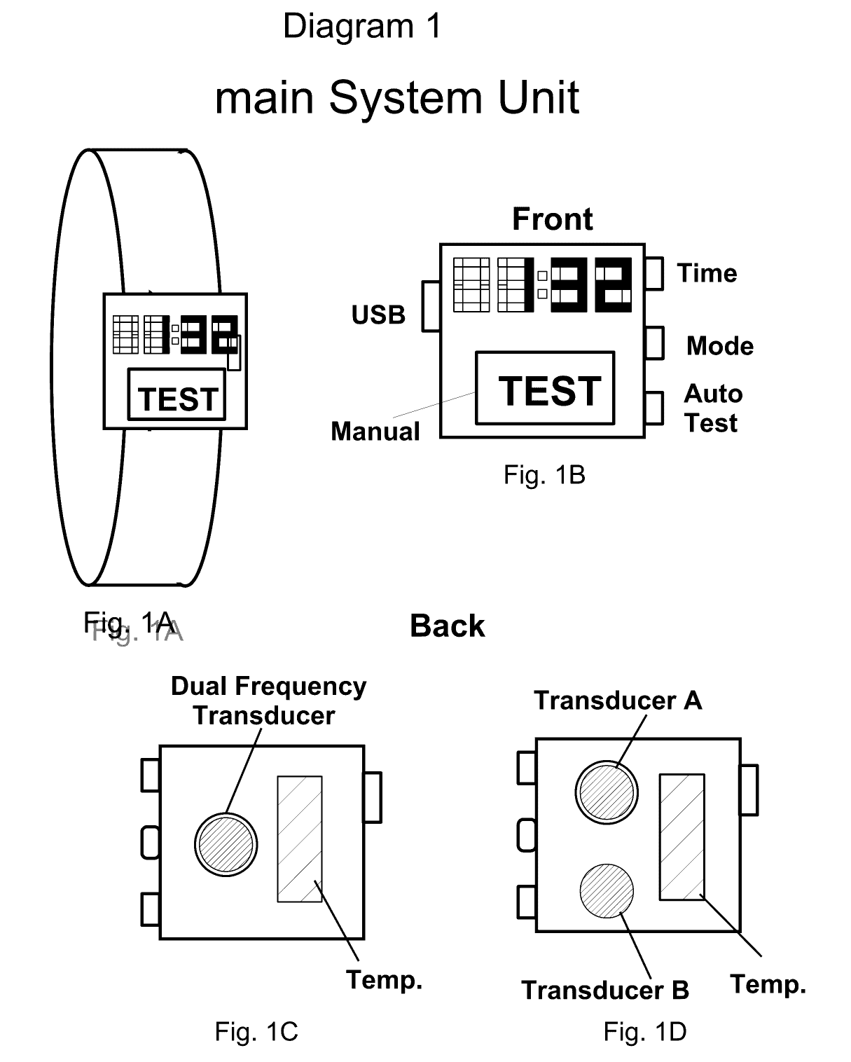

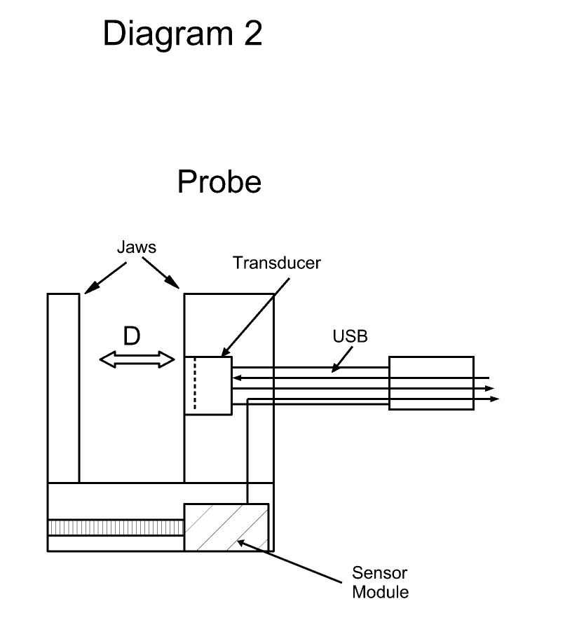

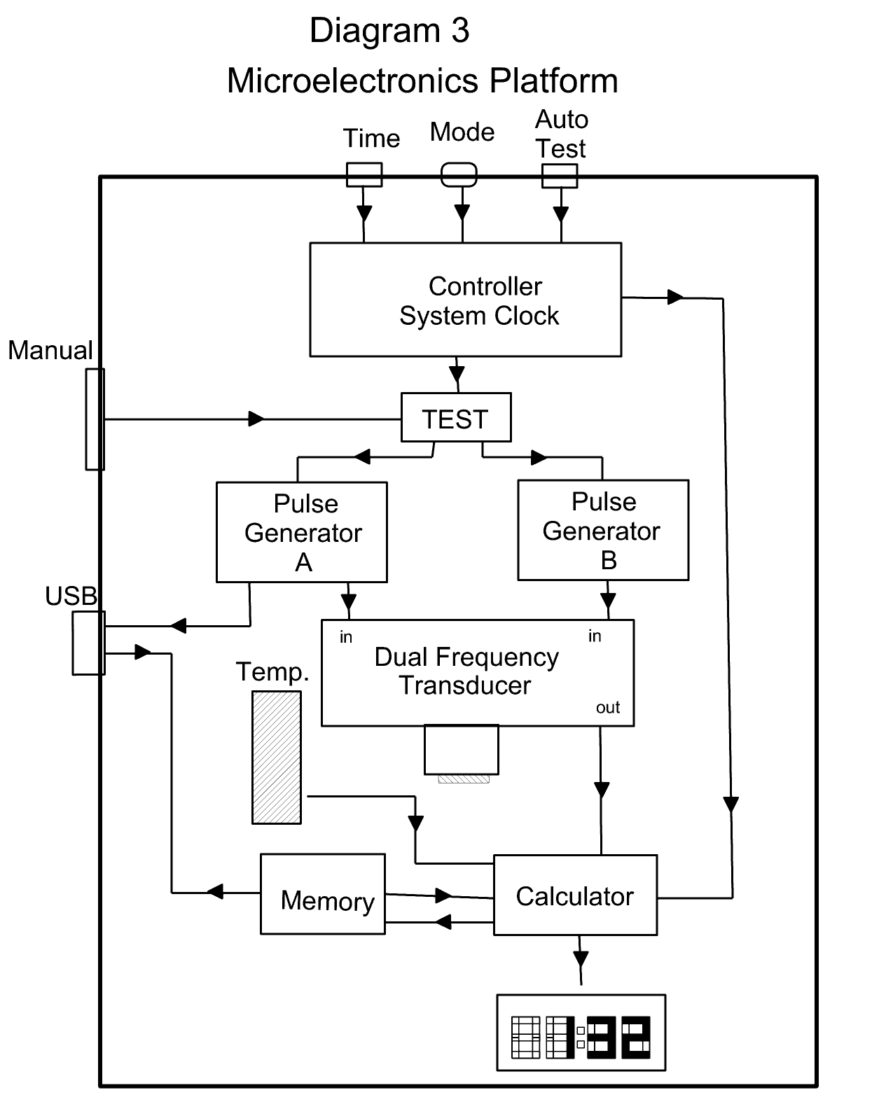

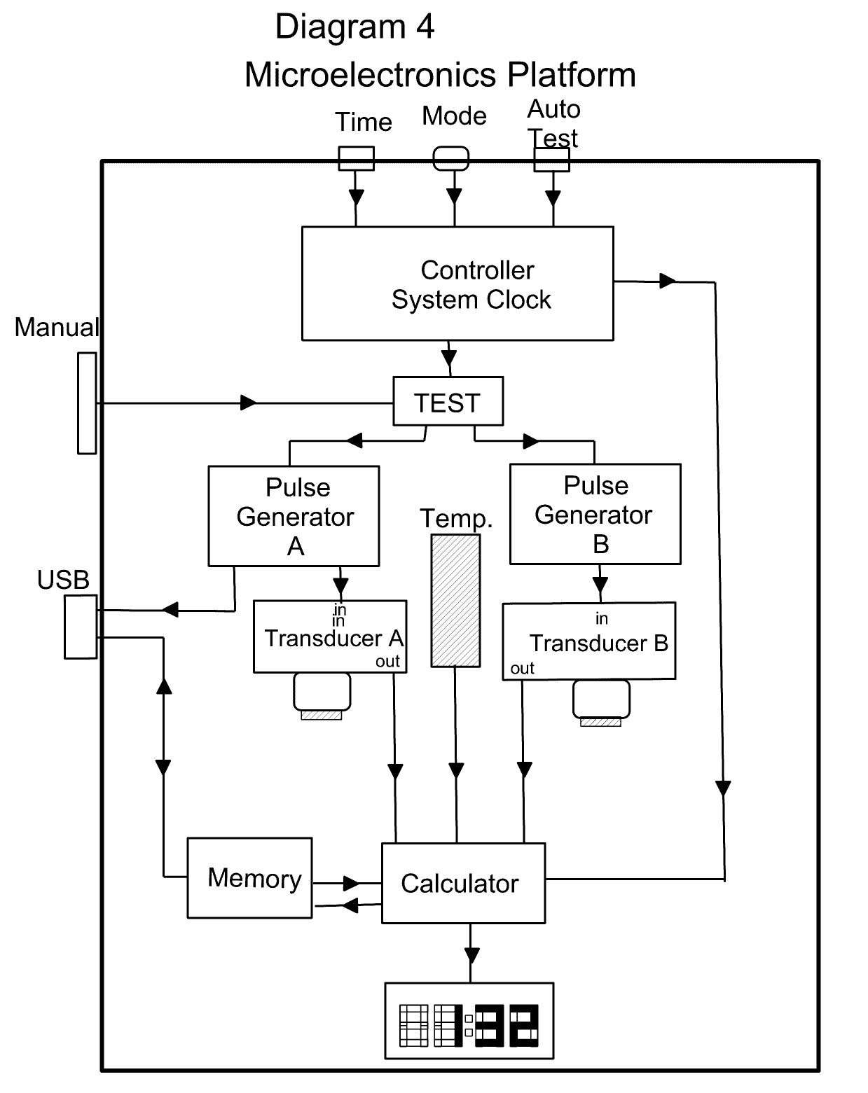

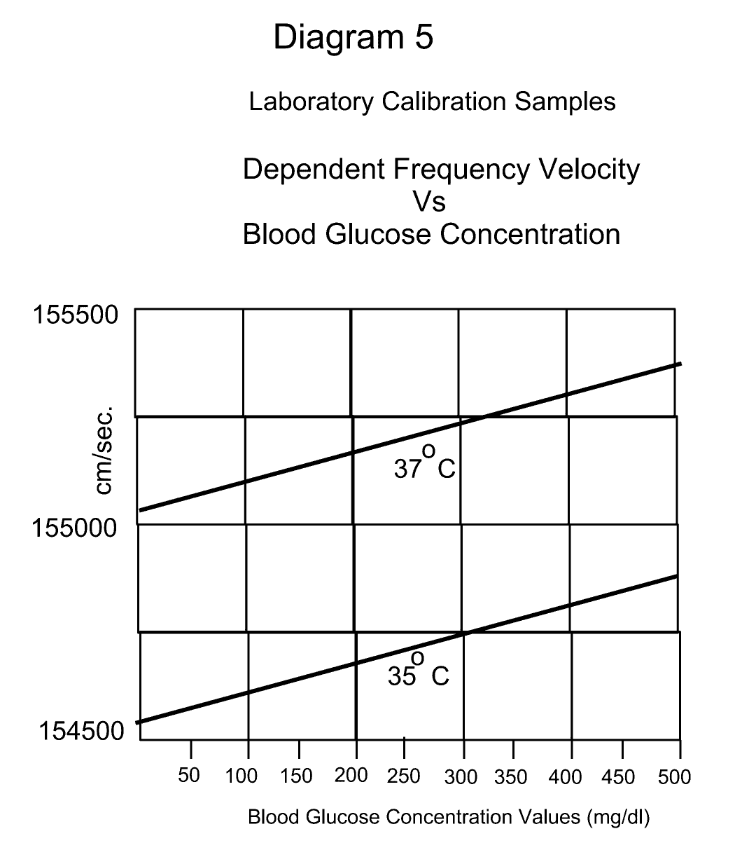

Diagram 2 shows the Probe component configuration.
Diagram 3 shows the microelectronics platform for the dual frequency transducer configuration within the main System Unit.
Diagram 4 shows the microelectronics platform for the two separate transducer configurations within the main System Unit.
Diagram 5 shows the matrix relationship of velocity versus blood glucose concentration values as determined by laboratory-prepared samples.

We claim:

1. A non-invasive, self-contained, wearable system for the real-time measurement of blood glucose concentration in tissue comprising:
means for two major components, a main System Unit and a Probe subsystem, whereby the measurements of two different ultrasound frequency waveform propagation times are utilized for determination of their respective velocities for subsequent calculation of glucose values:
means for measuring the propagation times of each ultrasound frequency waveform for calculation of their respective velocities:
means for determining the value of the independent frequency velocity, base velocity due to the Skin Effect utilizing a Probe subsystem:
support means for utilizing the Probe-defined base velocity for measuring the value of propagation distance referenced to the main System Unit:
means for determining the dependent velocity using the independent frequency velocity-defined distance value and measurement of the dependent velocity propagation time:
support means for a calibration matrix using laboratory-prepared samples for relationship of ultrasound dependent frequency velocities (cm/sec) versus blood glucose concentrations (mg/dl).

2. The non-invasive blood glucose monitoring system set forth in claim 1 containing a subsystem Probe comprising;
means for physical measurement of tissue thickness using electronic caliper jaws, whereby one jaw houses an ultrasound transducer and the other jaw provides the acoustic signal reflecting plate:
means for timing the travel time of the ultrasound signal through the tissue thickness:
means for calculating the frequency waveform velocity using the thickness distance value and signal travel time:
means for communication between the Probe and the main System Unit for data collection and processing.

3. The non-invasive blood glucose monitoring system set forth in claim 1 containing a main System Unit comprising:
means for a microelectronics platform for controlling all system functions, external communications, data storage and retrieval, frequency pulse generators, parameter calculations, display and power source.
